(19) United States
(12) Patent Application Publication
(10) Pub. No.: US 2007/0086918 A1
(43) Pub. Date: Apr. 19, 2007

(54) Cytometer

(76) Inventors: Lee F. Hartley, Calgary (CA); Orly Yadid-Pecht, Haifa (IL); Karan V.I.S. Kaler, Calgary (CA)

Correspondence Address: DYKAS, SHAVER & NIPPER, LLP, P.O. BOX 877, BOISE, ID, 83701-0877, US

(21) Appl. No.: 11/278,173

(22) Filed: Mar. 31, 2006

Related U.S. Application Data
(60) Provisional application No. 60/667,117, filed on Apr. 1, 2005.

(51) Int. Cl. G01N 33/00

(52) U.S. Cl. 422/73

(57) ABSTRACT

A real-time digital cytometer on a chip system utilizing a custom near field CMOS active pixel intelligent sensor that is flip-chip attached to a fluidic microchannel etched in a thin glass substrate. The CMOS active pixel sensor, fabricated using a 0.18 micron process, is a mixed signal chip comprising a sixteen pixel linear adaptive spatial filter coupled to a digital serial interface. This near field hybrid digital sensor topology obviates the need for both high resolution analog to digital conversion as well as conventional microscopy for the realization of real time optical cytometry. The custom sensor based design approach affords efficient scaling into a tiled multi-channel sensing configuration. The complete system, supported by a handheld graphical user interface and control module, demonstrates a viable micro total analysis sub-system for sample preparation and analysis which can support a wide range of applications ranging from cytometry to cell growth kinetics and analysis and various forms of fluid and droplet metering on an integrated and compact microfluidic platform.

### CROSS REFERENCE TO RELATED APPLICATIONS

This application claims the benefit under 35 USC 119(e) of provisional patent application No. 60/667,117 filed Apr. 1, 2005, the content of which is incorporated by reference herein.

### BACKGROUND OF THE INVENTION

A cytometer is a device that is employed to examine, count and subsequently sort microscopic sized particles, such as intact biological cells. In flow cytometry, such single particles, suitably tagged with fluorescence markers and immersed in an aqueous media are hydrodynamically focused by the sheath flow and made to traverse a small region of space illuminated by a focused laser beam. Depending on the optical characteristics of the cell traversing the focused coherent beam, and the chemo-optic markers it carries, the incident laser light will be scattered (forward scatter and backscatter) and/or induce floresence of the chemo-optic markers generated lightof different frequency. One or more of the optical signals emitted from the tagged particles are then collected as spectral bands, of predominantly visible light. Using chromatic filters, photomultipliers and analog to digital conversion, the acquired optical signals from individual cells can thus be used to identify and quantify the biophysical or biochemical characteristics of the cell sample population. Such cytometric devices have been shown to be capable of both high speed and sample throughput. For example, some such commercial systems, employing fluorescence activated cell analysis and/or sorting (FACS) can analyze particles at rates up to ˜100,000 particles per second. Additionally, the principle of cell sorting in flow cytometry allows this technology to be employed to affect physical separation of certain subpopulations of particles/cells from a heterogeneous mixture in an automated fashion. Thus fluorescence activated cell sorters (FACS) represent an incumbent technology in clinical cytometry. While commonplace in larger microbiology centers, FACS machines are large and expensive pieces of equipment which typically require highly trained personnel for succesful, utility, including operation and maintenance. The technology combines flow cytometry, fluorescent tagging and a reliance on electrostatic particle charging and their subsequent characteristic deflection in electric field to achieve both counting and fractionation of a heterogenous cell population into purer sub-populations.

Previously, a number of devices have explored miniaturization of various types of cytometeric devices. For example, Fu [Anne Yen-Chen Fu. Microfabricated Fluorescence-Activated Cell Sorters (uFACS) for Screening Bacterial Cells. PhD Thesis, California Institute of Technology, 2002] reported on a micro scale fluorescence activated cell sorter (μFACS) embodying a micro-channel T-junction etched in glass and employing laser illumination, beam splitters, optical objective gain, photomultipliers and high voltage actuation electronics. Providing affordable flow cytometry alternatives defines an area in which μTAS devices that can play a significant role by providing on-chip sample preparation and analyis of cells. Seeking alternatives to conventional microscopy, which impacts portability and cost, various topologies utilizing passive micro-channels, waveguides, fiber optics and/or integrated photo-detectors have been demonstrated to measure forward and/or side scattered radiation as particles moving through microfluidic channels interact with the incident illumination [Z. Wang, et al. Measurements of scattered light on a microchip flow cytometer with integrated polymer based optical elements. The Royal Society of Chemistry. Volume 4, 372-377, 2004; L. Fu and R. J. Yang and C. Lin and Y. Pan and G. Lee. Electrokinetically driven micro flow cytometers with integrated fiber optics for on-line cell/particle detection. Analytica Chimica Acta. 507:163-169. 2004; K. Singh and C. Liu and C. Capjack and W. Rozmus and C. Backhouse. Analysis of cellular structure by light scattering measurements in a new cytometer design based on a liquid-core waveguide. IEEE Proc. Nanobiotechnology. 151(1):10-16. 2004; V. Namasivayam, et al. Advances in on-chip photodetection for applications in miniaturized genetic analysis systems. Journal of Micromechanics and Microengineering. 81-90. 2004; P. LeMinh and J. Holleman and J. Berenschot and N. Tas and A. van den Berg. Monolithic Integration of a Novel Microfluidic Device with Silicon Light Emitting Diode-Antifuse and Photodetector. ESSDERC, 2002, 451-454.]. Nieuwenhuis has reported on two near-field optical sensors in a 1 μm bipolar process for particle shape based flow cytometric measurements [J. Nieuwenhuis and J. Bastemeijer and A. Bossche and M. Vellekoop. Near-Field Optical Sensors for Particle Shape Measurements. IEEE Sensors Journal. 3(5):646-651. October 2003]. The first such sensing device consists of a two photodiode strip-sensor which requires particle(s) to pass over the geometric center of the photodiode structure and thus requiring very precise hydrodynamic focusing for proper operation. The second device consists of a 2×20 photodiode array which, although capable of improved resolution, is plagued by a high input-output (I/O) bonding pad count and is furthermore challenged by the vast amounts of unconditioned analog data, that must be processed off chip in real time to demonstrate practicle utility. Thrush has described early work on an integrated VCSEL emitter, photodiode and lens system capable of providing optical coupling to microfluidic channels [E. Thrush, et al. Integrated Semiconductor Vertical-Cavity Surface Emitting Lasers and PIN Photodetectors for Biomedical Fluorescence Sensing. IEEE J. of Quantum Electronics. 40(5):491-498, May 2004]. Manaresi describes various topologies and system architectures for in-vitro manipulation and detection of suspended particles [N. Manaresi and A. Romani and G. Medoro and L. Altomare and A. Leonardi and M. Tartagni and R. Guerrieri. A CMOS Chip for Individual Cell Manipulation and Detection. IEEE JSSC. 38(12):2297-2305. December 2003]. However, these devices tend to be complex, with complex off chip processing. Thus there is good oppurtunity and room for further advancement in the field of microcytometers.

### SUMMARY OF THE INVENTION

There is provided in one embodiment of the invention a power transfer device that forms platform for integrated microfluidic cytometry, where optical image sensing and associated analog to digital processing circuits are integrated to the microfluidic substrate. This approach achieves near field optical coupling of the optical sensors to the contents of microfluidic channels or other flow paths for monitoring cells, or other suspended microscopic particles transported in microchannel fluid media.

There is also provided a power transfer device according to an embodiment of the invention that uses an array of photodetectors arranged transversely to a flow path, the photodetectors being oriented to receive radiation passing through the flow path. A processor is connected to receive electrical signals output from the photodetectors, and is configured to average the output of the photodetectors to generate an average, compare the output of each photodetector with the average and output signals indicating whether the output of each photodetector is above or below the average.

There is also provided according to an embodiment of the invention a micro-channel cytometer, comprising a substrate defining a flow channel; an array of photodetectors arranged transversely to the flow channel, the photodetectors being oriented to receive optical radiation transmitted through the flow channel; and an analog to digital processor connected to receive electrical signals output from the photodetectors. Advantageously, in one embodiment, each of the substrate, the array of photodetectors and the analog to digital processor are layered within a microfluidic chip. The analog to digital processor is preferably configured to sample output of the photodetectors, average the output of the photodetectors to generate an average signal, compare the output of each photodetector with the generated average signal and output a binary bit comprising N values, each ith value representing whether the output of the corresponding ith photodetector is above or below the average signal in real-time.

In accordance with a further embodiment of the invention, the cytometer may futhermore provide the necessary feedback to function as a closed loop optical control system. Combination of onboard digital signal processing in a microfluidic chip eliminates the need for fiber optics cables, photomultiplier collection, analog to digital conversion and conventional microscopy for it's operation. Onboard digital processing ensures that the system can be readily miniaturized for portability as compared to more conventional cytometry systems. The approach also addresses issues of μTAS manufacturability, affordability and repeatability. Multi-chip-module (MCM) based hybrid integration and advanced packaging technologies stand to reduce costs and enhance reliability of devices in the μTAS or fluidic microsystems domain. The cytometer may also provide disposable utility in applications that cannot tolorate sample cross contamination. To enhance such disposability, the digital processor may be designed as a custom CMOS integrated circuit. Furthermore, microfluidic assembly technologies can further ensure system cost and function can meet the stringent demands of biological and medical devices.

### BRIEF DESCRIPTION OF THE DRAWINGS

Preferred embodiments of the invention will now be described with reference to the figures, in which like reference characters denote like elements, by way of example, and in which:

FIG. 1 is a schematic showing integrated digital cytometer system components and architecture according to an embodiment of the invention catering to closed channel microfluidic sensing;

FIG. 2 is a schematic of an embodiment of a linear active pixel CMOS sensor chip for use in the embodiment of FIG. 1;

FIG. 3 shows one chain of an analog to digital processor for use in the sensor chip of FIG. 2;

FIG. 4 is a schematic of linear sensor architecture according to an embodiment of the invention digitally interfacing an adaptive spatial filter microchannel sensor chip to a microcontroller employing optical feedback control of chamber illumination;

FIG. 5 is an electrical schematic showing active pixel circuit topology with 7 μm square photodiode and p-channel reset device for use in the processor of FIG. 3;

FIG. 6 is an electrical schematic showing a linear sensor correlated double sampling circuit for use in the processor of FIG. 3;

FIG. 7 is an electrical schematic showing a linear sensor spatial filter circuit for use in the processor of FIG. 3, and which combines individual pixel outputs into global average and affords per-pixel digital offset control for compensation of system level fixed pattern nonuniformity;

FIG. 8 is a spatial filter truth table that defines each bit of the output of the spatial filter of FIG. 7 according to each pixel's relationship to the global average of all pixels;

FIG. 9 shows particle sensing as a result of near field optical shadowing of the active pixel array;

FIG. 10 shows output of a spatial filter according to the invention as emitted optical illumination is translated across the sensor active area from “BOTTOM”; and

FIG. 11 shows an embodiment of a cytometer with an open flow path.

### DETAILED DESCRIPTION OF PREFERRED EMBODIMENTS

In the claims, the word “comprising” is used in its inclusive sense and does not exclude other elements being present. The indefinite article “a” before a claim feature does not exclude more than one of the feature being present. A cytometer is a device for counting a flow of particles, for example particles moving in or transported by a stream of fluid. The particles may be any particles, and may be microscopic particles such as cells or may be individual liquid droplets. The particles may also be macroscopic objects such as vehicles. Electrical connections between block components are represented by lines in the drawings, but will be understood to represent conventional connectors available from electronics manufacturers, or patterned chip electrical connections formed in a conventional manner.

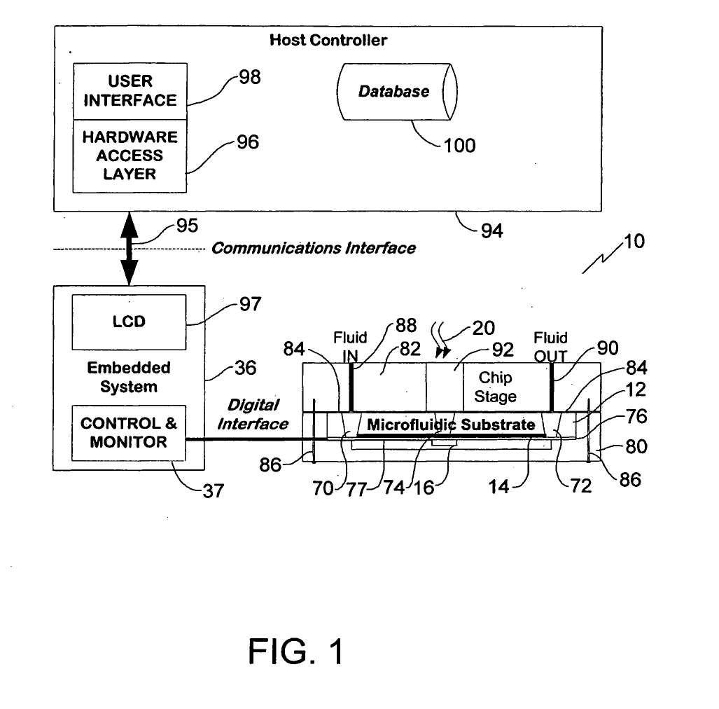

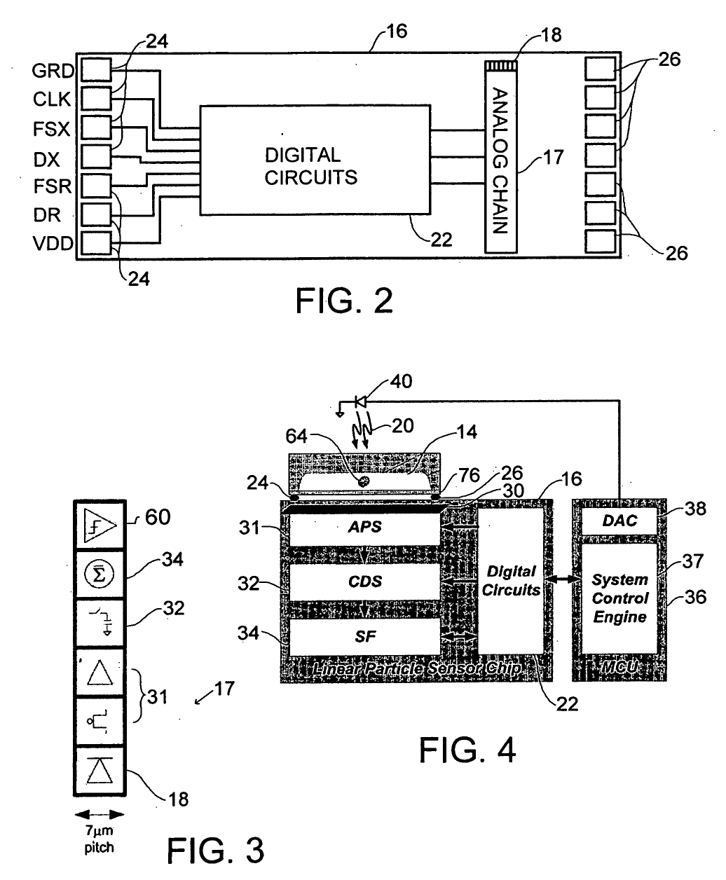

A schematic diagram of a digital cytometer 10 is illustrated in FIG. 1. The cytometer 10 comprises a muli-layered microfluidic chip. In the several layers of the cytometer 10 are a microfluidic substrate 12 defining a flow channel 14, and a sensor chip 16 that incorporates an array of photodetectors 18 (FIG. 2), which are preferably in a linear array. The photodetectors 18 are arranged transversely to and directly below the flow channel 14 and are oriented to receive radiation 20 passing through the flow channel 14. The radiation is electromagnetic radiation, such as but not restricted to optical radiation, or any suitable radiation that is detectable by the photodetectors 18. The sensor chip 16 also incorporates analog to digital processing chains 17, one chain 17 (FIG. 3) for each photodetector 18, and a digital processor 22 connected to receive digital electrical signals output from processing chains 17. The sensor 16 may use CMOS technology, and may be coupled to the microfluidic substrate 12 via flip-chip-on-glass assembly for near field particle detection. The digital processor 22 interfaces to a microprocessor 36, which provides an electrical control and monitoring subsystem for the sensor chip 16. The cytometer 10 also includes a conventional mechanical fluid processing system for supplying fluid carrying cells to the microfluidic channel 14, parts of which are shown in FIG. 1. The channel need not be closed, and may be any path along which particles flow.

The sensor 16 may be made using 0.18 μm CMOS technology, and is preferably designed and fabricated for flip-chip attachment and integration on glass aboard the microfluidic substrate 12. As shown in FIG. 2, the sensor 16 is provided with input/output active electrical bonding pads 24. A low input/output electrical pad count such as seven is obviously desirable to minimize the implementaion chip area. The sensor 16 is also provided with seven mechanical pads 26. The pads 26, being located at one end of the sensor 16, facilitate reliable and straight forward microfluidic channel integration employing either flip-chip bonding or more conventional wire bonding techniques. An exemplary sensor 16 has characteristics as follows: Height 1.0 mm; Width 2.4 mm; Thickness 0.7 mm; Supply Voltage 1.8V; Power Consumption 15 mW; IO Pads consisting of 5 digital and 2 power supply; Pixel size 7 μm; and Number of Pixels 16. With a channel width of 112 μm, and pixel size of 7 μm, a convenient 16 pixels may be aligned across the channel 14. An approximately 100 μm channel width is typical of microfluidic operations, and in general the channel width may vary from 30 μm to 300 μm. A pixel size of 7 μm is most convenient for detecting cells having a size in the order of 5-10 μm, however this may be increased to accommodate larger cells without impacting device opaeration. The pads 24 may include the following signal connections: ground (GRD), clock (CLK), a voltage source (VDD such as 1.75 to 1.85 V) and a serial digital bit streams FSX (transmit frame sync), DX (transmit data), FSR (receive frame sync) and DR (received data). The bit stream pads are preferably diode clamped to VDD and GRD to minimize electrical damage to the on-chip circuitry due to electrostatic discharge or other over volatge condition generated externally.

There may be more than one sensor 16. A pair of sensors 16 may be placed with their arrays of photodetectors side-by-side to form a two-dimensional array of photodetectors 18. In another embodiment, two or more sensors 16 are placed apart from each other, but each with their photodetectors 18 arranged transversely across the channel 14. Such a configuration permits determination of particle velocity. One or more sensors 16 placed in various positions within a microfluidic system can serve as feedback sources for control information in microfluidic systems by providing real time flow control information at fluidic ports or at key points throughout interconnected microfluidic networks.

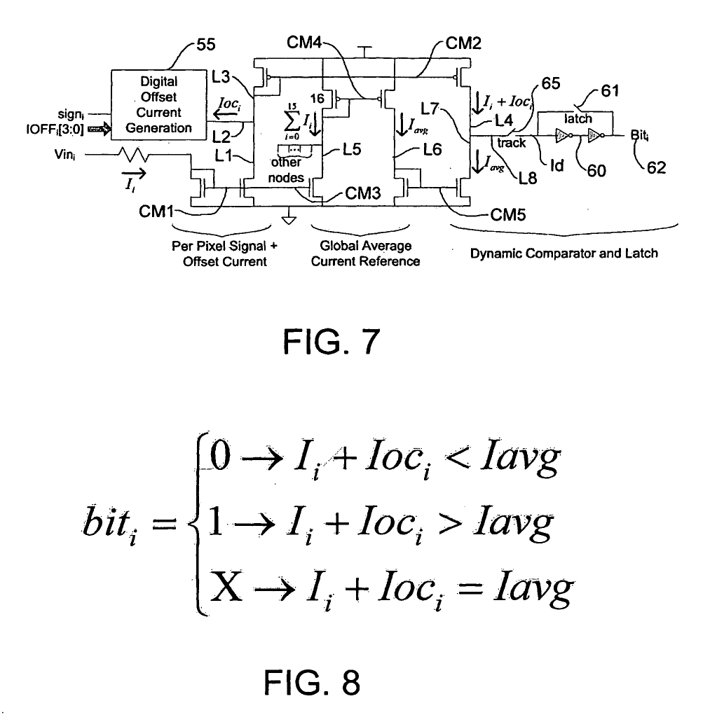

A block diagram of an embodiment of the sensor 16 architecture is depicted in FIGS. 3 and 4. The photodetectors 18 are incorporated within an array 30 of linear active pixel sensors (APS) 31, the active pixel sensors 31 of which output to a sampler 32, which may be for example a correlated double sampler (CDS). The sampler 32 outputs to an adaptive spatial filter (SF) 34. Also shown in FIG. 4 is a feedback system incorporating a microcontroller unit 36, digital-to-analog converter 38 and light emitting diode 40. The diode 40 provides illumination 20 of the channel 14. The digital processor 22 may incorporate a programmable digital offset control circuit 55 (FIG. 7) to facilitate compensation for fixed pattern effects such as pixel-to-pixel process variations and inhomogeneous near-field illumination present in the convolved optical system of: emitter profile, sensor surface, fluidic media, chamber geometry and the material used to under fill the flip-chip sensor after bonding. The sensor chip 16 is interfaced to and controlled by microcontroller 36, for example a low power, battery operable microcontroller platform. The sensor 16 provides real-time particle population and count statistics under a variety of continuous or circulating flow conditions present in the microfluidic channel 14 housed above the sensor 16.

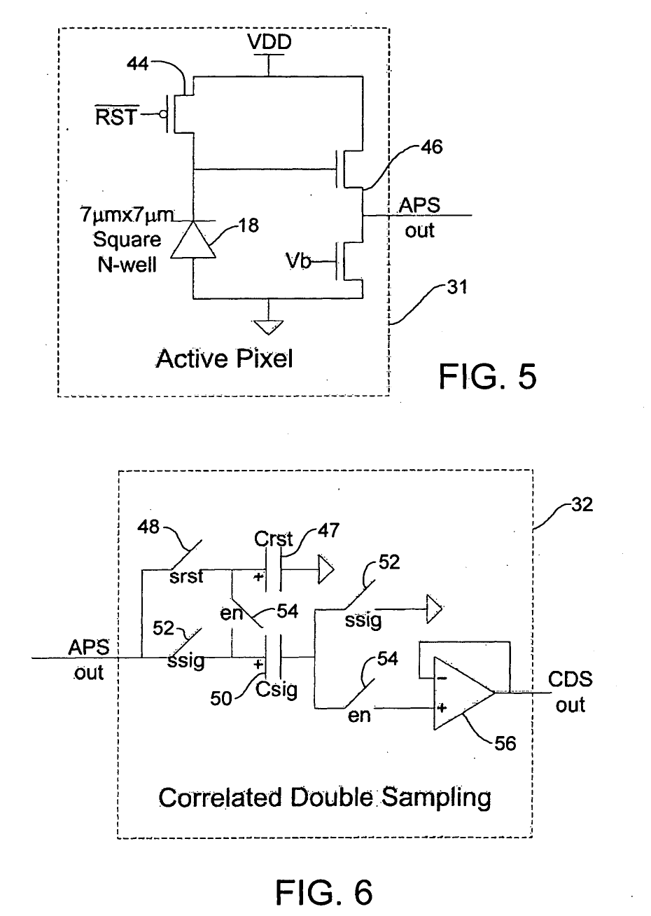

Each active pixel sensor 31 in each analog to digital chain 17 may have the design shown in FIG. 5. In FIG. 5, a photodiode 18 may for example be a 7 μm×7 μm square N-well provided with a reset 44. Power is supplied by VDD, and the output of the active pixel sensor 31 is supplied by buffer amplifier 46. The APS array 30 provides flexibility in the second dimension with respect to fill factor (defined as the ratio of active area/total pixel area). The per-pixel signal path layout is achieved on a 7 μm channel pitch yielding a 90% fill factor across the array 30. The in-pixel p-channel reset device 44 provides increased pixel dynamic range by raising the photodiode reset voltage at the start of each integration cycle. After reset, the voltage applied to the diode 18 is a maximum. As light falls on the diode 18, the diode 18 drains, reducing the voltage during an integration cycle. With a fixed reset, the diode 18 always returns to the same voltage maximum.

The output of each active pixel sensor 31 in the array 30 is connected to a single ended correlated double sampling (CDS) circuit 32, as shown in FIG. 6. The operation of the CDS circuit 32 is as follows. After the application of reset to the active pixel's photodiode 18 at the start of an integration cycle, the analog pixel output voltage (Vrst) is sampled and its value stored on Crst sampling capacitor 47 by the pulsed assertion of the signal srst through switch 48. At the end of the integration period, the analog pixel output voltage (Vsig) is sampled and stored on the Csig capacitor 50 by the pulsed assertion of the signal ssig through switch 52. Finally, the en signal is asserted through switch 54 to steer the voltage Vrst-Vsig to the input of a unity gain folded cascode amplifier 56. The buffered output signal (CDSout) is subsequently used by the spatial filter circuit 34 for final frame processing.

The adaptive spatial filter 34 (including digital offset control) is connected as an integral part of the chip 16 containing the active pixel sensors 30 and is connected as part of the analog to digital chain 17. The spatial filter output is serially accessible as a 16-bit digital word.

The exemplary spatial filter 34 shown in FIG. 7 is connected as a group of current mirrors CM1, CM2 etc. Each current mirror CM comprises a pair of transistors, for example MOSFETs, connected gate-to-gate in conventional fashion, and each current mirror is identified in FIG. 7 by the line connecting the gates of the MOSFETs in the current mirror. In FIG. 7, the voltage (Vini) represents the output CDSout of the unity gain output buffer 56 of the ith correlated doubling sampling circuit 32 (FIG. 6). From this per-pixel signal voltage, three reference currents are generated: the signal current (Ii), a digitally programmable offset current (Ioci) and the average of all of the sixteen signal currents (Iavg). Current mirror CM1 generates current Ii in line L1. Digital offset current generator 55 produces current loci in line L2, and the combination of Ii and Ioci appears in line L3. Current mirror CM2 causes current Ii+Ioci to appear in line L4. Current mirror CM3 causes Ii to be added to line L5 where it combines with currents Ii from the other samplers 32 in the chains 17. The sum of the currents Ii is then mirrored by current mirror CM4 to appear in line L6, and further mirrored by current mirror CM5 to appear in line L7. A difference current Id=Ii +Ioci−Iavg, then appears in line L8.

During the tracking phase, the output comparator 60 generates a digital bit 62 whose value is set according to the truth table conditions shown in FIG. 8. The spatial filter truth table in FIG. 8 defines each bit (0or 1) of the spatial filter output word according to each pixel's relationship to the global average of all pixels, that is, according to whether the value of CDSout for each active pixel sensor in the array 30 is above or below the average Iavg .

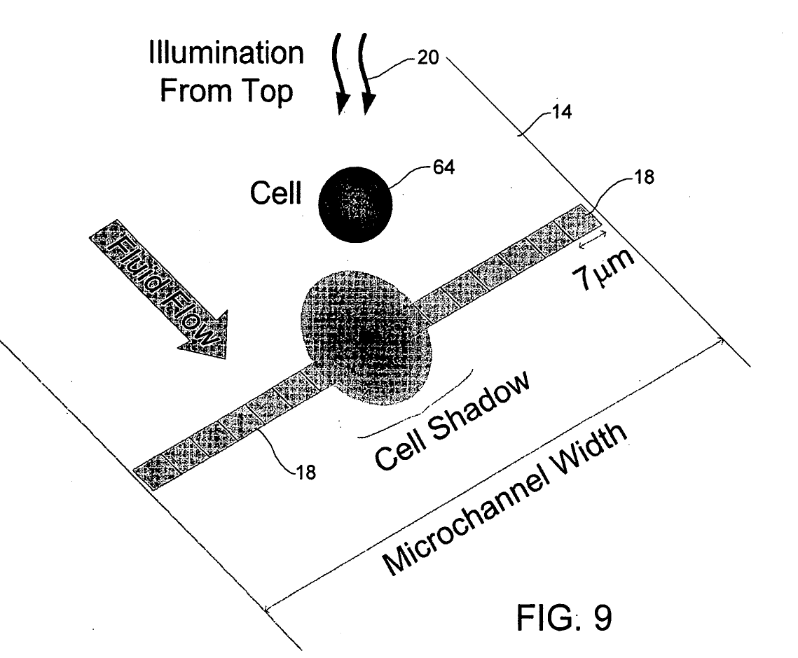

The operational behavior of the spatial filter 34 is described as follows. At the end of each integration period, the digital processor 22 operates latch 61 to latch the result of the spatial filter truth table calculation for each pixel 31 in the array 30 and stores the digital result into a sixteen bit serial shift register formed as part of the digital processor 22. The corresponding bit of each sensor pixel 31 in the resulting sixteen bit double word is an indicator of whether the illumination of that pixel 31 is above or below the value of global average of all pixels 31 in the array 30. This sixteen bit digital word can be programmatically monitored in real time to detect changes in the frame to frame readout as particles 64 momentarily shadow (FIG. 9) one or more of the active pixels 31 in the array 30.

Thus, in the spatial filter 34, the output bit of each pixel 31 in the resulting word is a function of the illumination of the pixel 31 as well as the average illumination of all the pixels 31. A particle 64 need not pass directly over a particular pixel 31 in order to illicit a change in the state of that pixel 31 in the resulting word. This dynamic characteristic of the spatial filter 34 makes possible a powerful mode of operation that is extensively exploited in the current embodiment, as described below. The microcontroller 36, through feedback, first trims the background illumination level by varying the output of diode 40 such that at least one of the pixels 31 in the array 30 is consistently at or very near the average pixel output. This state can be detected easily when the resulting digital bit of a particular pixel 31 becomes random frame to frame. At this point, the dynamic comparator 46 associated with this trapped pixel 31 is pinned near the high gain transition point of its input output transfer characteristic. At this highly sensitive operating point, even a very small perturbation on the analog output voltage of the pixel 31 or on the average pixel output of the array 30 results in a strong bias of the digital output for the trapped pixel 31 during subsequent frames.

The count of historical ones and zeroes in the digital bit stream of the sensor 16 may be accumulated and tracked either within the digital processor 22 or the microcontroller 36. To begin accumulation and tracking, a signal is sent from the digital circuits 22, under control of microcontroller 36, to track enable switch 65. The passage of particles over the sensor 16 can be detected when the free running output bit stream associated with such a trapped pixel 31 deviates from the baseline by a calculable threshold amount. This signal processing approach affords a significant advantage over conventional spatial filter operation. Operation of the spatial filter 34 is differential in nature. The spatial filter 34 may detect either an increase or a decrease in illumination of target pixels 31. In contrast, conventional spatial filters used for star field gazing are frequently only interested in the brightest object and are less concerned with transient frame to frame variations in those intensities. As such, conventional spatial filters typically only operate in a single ended mode, whereas the linear particle sensor described here is designed for dynamic differential operation.

Digital offset compensation may be used to alter the threshold at which individual pixels 31 change states. It has been found that with the sensor 16 under little or no illumination condition, the deviation between pixels 31 is very small with no pixel requiring an applied offset beyond ±2 to flip states. With increasing illumination intensity, the analog signal chain displays a flat region throughout which pixels 31 in the array 30 demonstrate a wider variation (range −4 to +7) in order to toggle. As intensity is further increased, causing elements in the analog signal path to saturate, the minimum offset value to toggle each pixel's state collapses back to the “no-signal” levels. Above this intensity level, no further changes are observed. From this data, the linear region or input dynamic range of the sensor 16 may be determined. Some pixels 31 have been found to demonstrate a change (at very low intensity levels) in the polarity of the offset required to flip their state. Some of the pixels start out requiring a positive offset current (to flip a 0 to a 1) and end up requiring a negative offset current (to flip a 1 to a 0); and visa versa. These pixels are well suited (even without any digital offset compensation) for being pinned at their transition points by the illumination feedback control loop for use in a dynamic averaging scheme.

Digital offset compensation may be carried out by any of a number of ways. During a calibration phase, the controller 36 may instruct the diode 40 to illuminate the channel 14 in the absence of particles. The controller 36 then instructs the control circuits 22 to read the output of each sampler 32. If it is found that the output of a sampler 32 is too far from the average for the sampler 32 to have useful output, the controller 36 computes an offset current that would bring the corresponding sample output closer to the average current from the samplers 32. The computed value for the digital offset may be supplied as positive value IOFFi[3:0] indicating the magnitude of the offset plus a signal signi indicating the sign of the offset. The digital offset generator 55 then generates the appropriate offset current loci using any of a number of suitable means such as through four bit p-channel digital to analog current mode converters and complementary n-channel converters. The offset current range may be chosen to cancel +/−25% of the background non-homogeneity about the average level. The range may also be scaled with the average signal intensity entering the spatial filter.

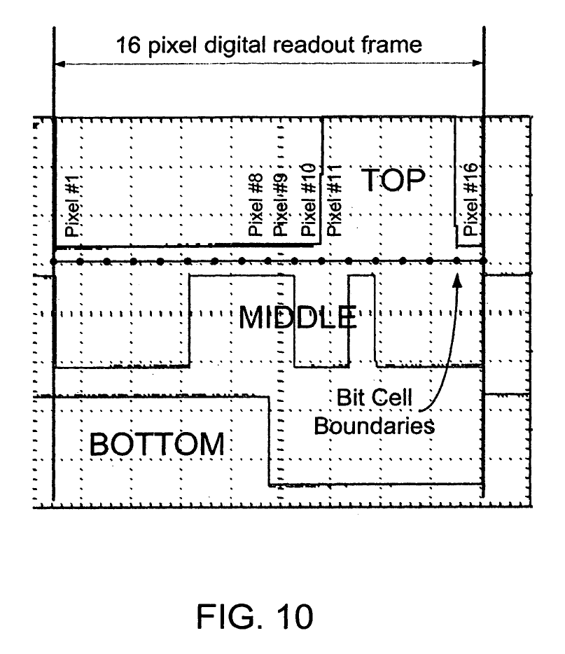

FIG. 10 shows exemplary waveforms as a 100 μm moveable pinhole aperture placed approximately 500 μm atop of the sensor with uniform illumination was translated horizontally across the pixel array 30 as the sensor output was monitored. The digital output word was captured by an oscilloscope for three static positions of the pinhole: oriented over the bottom, middle and top of the sensor active area as viewed through the microscope field of view. The waveforms of FIG. 160 display the sensor's digital output word for each of the three arrangements as indicated by the labels: “BOTTOM”, “MIDDLE” and “TOP”. In the figure, the “BOTTOM” pixels map to the left side of the digital result word whereas the “TOP” pixels map to the right side. In the figure, two sensor offset effects are noted; one in the “MIDDLE” trace and one in the “TOP” trace. While the “MIDDLE” trace confirms a tendency for the “central” pixels in the array to be latched “high”, two pixels continue to be reported as being illuminated below the global average. The “TOP” trace, similarly, does not include an asserted “high” bit value for pixel sixteen. Such spatial filter inaccuracies can arise from offsets in the chip and/or the optical system providing the illumination as has been discussed earlier.

As shown in FIGS. 1 and 4, the flow channel 14 with flow ports 70, 72 and viewing port 74 may be formed in a glass substrate 12, such as may be formed by two Schott D263 glass sheets thermally bonded together. This sheet is thermally bonded to a sheet 76 to which the sensor 16 is bonded. The sheet 76 may be for example a 100 μm thick sheet of glass. Electrical connectors 77 for the sensor 16 are formed on the surface of the sheet 76 by patterning with metallization such as Cr—Ni—Au (50 nm-50 nm-1250 nm) metallization suitable for thermocompression or solder reflow flip-chip bonding of the sensor chip 16. In this metal stack, nickel may be employed as a barrier against brittle chrome-gold inter-metallic formation which is known to hinder bond effectiveness during both solder reflow and thermo-compression flip chip bonding. The substrate 12 provides mechanical stability, in addition to providing microfluidic channels and fluid ports. This layered microfluidic chip thus includes the flow channel and attached sensor. The substrate 12 contains the channel 14, which may be for example a 100 μm×100 μm channel, and the channel 14 passes transversely over the center of the linear active pixel array 30 such that the pixel array 30 spans the entire channel width. The sensor active area is exposed through a powder blasted through hole 74 in the substrate 12 which serves as both a clear optical path as well as an alternate particle injection port. The mechanical pads 26 and electrical pads 24 may be provided with tin lead solder to facilitate bonding. To avoid oxidation problem associated with Al pads a plasma ash procedure may be carried out prior to bonding. Bonding may be performed using a conventional flip chip bonder equipped with a solder reflow arm.

The port 74 for the access of illumination 20 to the channel 14 preferably provides for uniform or known intensity illumination profile across the channel, such as lambertian. The illumination 20 provided by diode 40 is preferably controllable. The port 74 should be wide enough to provide illumination to each part of the channel 14. The spatial filter 34 with digital offset control automatically compensates for variation of illumination across the channel.

The layered microchip forming the cytometer 10 may be housed in a two part chip carrier stage, for example milled in acetyl delrin, as shown in FIG. 1. A bottom piece 80 of the assembly forms a slide socket into which the layered microchip is recessed. Bottom piece 80 also provides mechanical stability and clearance slots for the underside flip chip mounted sensor 16 and the necessary electrical connections. Fluidic connection to the hybrid glass substrate 12 may be facilitated by an o-ring compression seal 84 formed between the glass substrate and a top chip stage piece 82. As the clamshell set screws 86 are tightened, the compressed O-rings 84 form a seal between macroscopic fluid ports 88, 90 coming through the upper clamshell piece 82 and the underlying microfluidic ports 70, 72. Illumination and viewing occurs through a large hole 92 in the top chip stage piece 82 which is aligned over the centre of the active area of the sensor 16.

Once sealed, operation of the system proceeds as follows. In free running mode (at chosen frame rate), the sensor digital bit stream is monitored while the illumination is programmatically ramped from darkness. As the intensity is ramped, different pixels 31 will display transition regions at different intensities during which a pixel's output is a stream of zeroes and ones with a calculable average value (the average duty cycle). The sensor 16 is run according to a sensor algorithm controlled by the microcontroller 36 and carried out by the digital circuits 22. In one example, a sensor interface algorithm uses digital FIFO queuing of incoming serial bit stream in order to digitally count particle passage events. For real time visualization, a digital to analog converter may be used to create an analog representation of the dynamic behavior of the sensor, which may be displayed on LCD 97. For every frame, the “current” pixel value (zero or one) is added to a running sum, and the pixel value is pushed into an N-deep FIFO. The Nth previous sample (zero or one) ejected from the FIFO is subtracted from the running sum. By adjusting the illumination via the optical feedback control a stable point is reached at which the running sum assumes an average value of N/2. While there is random variation around N/2, the instantaneous count can be considered a qm format number where m=log2 (N). For example, a 16-deep FIFO relates to a q4 number being generated:
Sum=0 if there are all 0 bits in last N samples

Sum=8 if there are 50% ones, 50% zeroes in last N samples

Sum=16 if there are all 1 bits in last N samples

As particles pass over the sensor surface, they disrupt the duty cycle of the output bit stream associated with pixels in the trapped mode of operation. This deviation manifests as a departure of the running sum from the average value, and this digital deviation may be compared to a digital threshold enabling the counting of these disruption events. The counting function may be carried out within the digital circuits 22.

The control engine 37 of microcontroller 36 controls the operation of the sensor chip 16. The control engine 37 may be for example an MSP430 microcontroller, and provides power commands, counter control commands, such as read and reset, integration time, and the application of digital offset signals. Other controls from the control engine 37 will depend on the functions carried out by the sensor chip 16. For example, particle size detection may be roughly based on the number of pixels obscured by a particle. The counter may be configured to count bright events rather than dark events. Particle velocity determination may take into account signals from two or more sensors 16. The sensor 16 may also be modified to detect fluorescence of particles that have been contacted with a fluorescent marker and exposed to light excitation. The particles within the microfluidic channel may be sorted by any of various means such as electrophoresis and dielectrophoresis. The microcontroller 36 will typically interface to a conventional computer 94 acting as a host controller through a conventional communications interface 95, via hardware access layer 96 and user interface (keyboard) 98. A database 100 may be formed by memory within the computer 94.

The control functions provided by control 37 depend on the application. As an example, write functions may use the FSX pad 24 on the chip 16 to program the digital offset generator 55, as well as to define the integration period and trigger the generation of new readout data. Sequential write operations may serially fill a 90-bit control register in the digital circuits 22. The control register may comprise 5×16=80 bits of offset information plus 10 reserved bits. Thus, the digital offset command is include in the 5 bit word, which may be asserted through the DX pad 24. In response to assertion of a signal on the FSX pad 24, the sensor 16 internally ends the integration period, generates a new digital spatial filter result and shifts that result out on the DR pad 24. The command signals sent by the control circuits 22 to the analog to digital chains 17 as a result of assertion of the FSX signal, include ssig applied to the samplers 32, followed by track and latch applied to the spatial filters 34, followed by reset applied to the active pixel sensors 31, and then srst applied to the samplers 32. Data is then read out as a 16 bit word from the output of the spatial filters 34 on the DR pad 24. The controller 37 may operated in a standalone mode with no host controller or may be controlled by host controller 94.

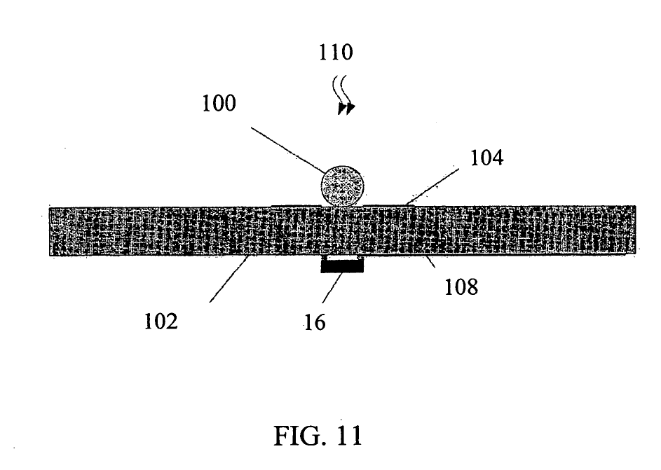

Referring to FIG. 11, there is a shown a cytometer that uses an open flow path. A particle 100 is shown located on a substrate 102. The substrate 102 may be any suitable substrate for the particle being considered. For example, where the particle 100 is a liquid droplet of small size, the substrate 102 may be made of PMDS or glass. Electrodes 104 may be deposited or secured on the substrate 102 by any suitable means and may be used to generate a motive force, such as by dielectrophoresis or electrophoresis, to cause the particle 100 to move across the substrate 102. Electric power for the electrodes 104 may be supplied by any suitable device. A pattern of electrodes 104 on the substrate 102 establishes a flow path for the particle 100, which in this case may be moving across the substrate in a direction perpendicular to the plane of the view. A sensor 16 is attached to a side of the substrate 102 opposite to the particle 100 for example by flip-chip bonding, or other suitable methods. Control signals and data signals to and from the sensor 16 to a controller (not shown, but may be the same as controller 36) are provided by electrical connectors 108, such as Cr—Ni—Au metallization patterned on the substrate 102. Illumination 110, as in the embodiment of FIG. 1, is supplied by a diode under control of the controller 36. The embodiment of FIG. 11 works in the same way as the embodiment described in relation to FIGS. 1-10, except that the flow path of FIG. 11 is confined electrically. The flow path in other embodiments may also be defined by gravity, as in the case of a falling body, or any other way of defining a flow path.

The described cytometer provides improvements in portable μTAS viability by incorporating near field optical sensing. This obviates the need not only for conventional microscopy, but also for precision analog to digital conversion in real time microfluidic particle sensing applications. Furthermore, the adaptive spatial filter architecture encapsulated by an all digital external interface relegates analog signal conditioning issues to the on-chip domain instead of the vastly more challenging system wide domain. Lastly, by leveraging commercially proven assembly technology, our approach also points to cost reduction opportunities through improved manufacturability and greater reliability. immaterial modifications may be made to the embodiments of the invention described here without departing from the invention.

What is claimed is:

1. A cytometer, comprising:
a substrate defining a flow path;
an array of photodetectors arranged transversely to the flow path, the photodetectors being oriented to receive radiation from the flow path;
an analog to digital processor connected to receive electrical signals output from the photodetectors; and
each of the substrate, the array of photodetectors and the analog to digital processor being layered within a microfluidic chip.

2. The cytometer of claim 1 in which the analog to digital processor comprises plural processing blocks, each processing block corresponding to one of the photodetectors in the array of photodetectors.

3. The cytometer of claim 2 in which each of the photodetectors forms part of an active pixel sensor.

4. The cytometer of claim 3 in which each of the processing blocks comprises a sampler connected to a corresponding active pixel sensor.

5. The cytometer of claim 4 in which the analog to digital processor comprises an averager that averages output from each of the samplers.

6. The cytometer of claim 5 in which each of the processing blocks comprises a comparator connected to compare output from the sampler in the processing block with output from the averager and generate an output representing whether the output of the sampler is above or below the average.

7. The cytometer of claim 6 in which the analog to digital processor comprises a latch for generating a binary output signal comprising the output of the comparators.

8. The cytometer of claim 1 in which the analog to digital processor is configured to sample output of the photodetectors, average the output of the photodetectors to generate an average, compare the output of each photodetector with the average and output a binary value bit comprising N values, each ith value representing whether the output of the corresponding ith photodetector is above or below the average.

9. The cytometer of claim 1 further comprising a radiation source oriented to illuminate the flow path.

10. The cytometer of claim 9 in which the microfluidic chip comprises a feedback circuit, the feedback circuit comprising a control for the radiation source, the control being responsive to output from the photodetectors.

11. The cytometer of claim 10 in which the analog to digital processor is configured to sample output of the photodetectors, average the output of the photodetectors to generate an average, compare the output of each photodetector with the average and output a binary value bit comprising N values, each ith value representing whether the output of the corresponding ith photodetector is above or below the average.

12. The cytometer of claim 11 in which the control is configured to adjust the radiation source to illuminate the photodetectors in which at least one of the photodetectors has an output close to the average.

13. The cytometer of claim 12 in which the control is layered within the microfluidic chip.

14. The cytometer of claim 1 in which the flow path is a channel in the microfluidic chip.

15. The cytometer of claim 14 in which the flow channel has a width of between 30 and 300 micrometers.

16. The cytometer of claim 1 in which the analog to digital processor is incorporated on a chip, and the photodetectors are aligned along one edge of the chip.

17. The cytometer of claim 1 in which the array of photodetectors is a linear array.

18. A cytometer, comprising:
a substrate defining a flow path;
an array of N photodetectors arranged transversely to the flow path, the photodetectors being oriented to receive radiation passing through the flow path;
a processor connected to receive electrical signals output from the photodetectors; and
the processor being configured to average the output of the photodetectors to generate an average, compare the output of each photodetector with the average and output signals indicating whether the output of each photodetector is above or below the average.

19. The cytometer of claim 18 in which the processor comprises an analog to digital processor that samples the output from the photodetectors.

20. The cytometer of claim 18 further comprising an optical radiation source oriented to direct light towards the flow path.

21. The cytometer of claim 20 further comprising a feedback circuit, the feedback circuit comprising a control for the optical radiation source, the control being responsive to output from the photodetectors.

22. The cytometer of claim 21 in which the control is configured to adjust the optical radiation source to provide an illumination level on the photodetectors in which at least one of the photodetectors has an output close to the average.

23. The cytometer of claim 18 in which the flow path is a channel in a microfluidic chip.

24. The cytometer of claim 23 in which the flow channel has a width of between 30 and 300 micrometers.

25. The cytometer of claim 18 in which the array of photodetectors is a linear array.

26. A cytometer, comprising:
an array of N photodetectors arranged transversely to a flow path, the photodetectors being oriented to receive radiation passing through the flow path;
a processor connected to receive electrical signals output from the photodetectors; and
the processor being configured to average the output of the photodetectors to generate an average, compare the output of each photodetector with the average and output signals indicating whether the output of each photodetector is above or below the average.

27. The cytometer of claim 26 in which the processor comprises an analog to digital processor.

28. The cytometer of claim 26 in which the array of photodetectors is a linear array.
